(19) United States
(12) Patent Application Publication
(10) Pub. No.: US 2012/0165735 A1
(43) Pub. Date: Jun. 28, 2012

(54) Devices and Methods for Reducing Electrical Noise in an Irrigated Electrophysiology Catheter System

(76) Inventors: Sandra Keh, Irvine, CA (US); Michael C. Bednarek, Irvine, CA (US)

(21) Appl. No.: 12/979,411

(22) Filed: Dec. 28, 2010

(51) Int. Cl. A61M 1/00

(52) U.S. Cl. 604/151

(57) ABSTRACT

An irrigation system for use with an irrigated electrophysiology catheter includes a peristaltic pump and an irrigation tube. The pump generally includes a pump clamp and a rotor, which are spaced apart to provide a tubing channel therebetween. The irrigation tube is inserted into the tubing channel. At least one of the pump clamp and the irrigation tube is treated to reduce surface adhesion therebetween. This advantageously minimizes perturbations to the double layer, which reduces a noise signal that might otherwise couple to and distort an electrogram signal.

### BACKGROUND OF THE INVENTION

a. Field of the Invention

The present disclosure relates to irrigated electrophysiology catheters. In particular, the present disclosure relates to devices and methods for reducing electrical noise in irrigated electrophysiology catheter systems.

b. Background Art

Catheters are used for an ever growing number of medical procedures. To name just a few examples, catheters are used for diagnostic, therapeutic, and ablative procedures. Typically, the physician manipulates the catheter through the patient's vasculature to the intended site, such as a site within the patient's heart. The catheter typically carries one or more electrodes (in the case of so-called “electrophysiology catheters”) or other diagnostic or therapeutic devices, which can be used for ablation, diagnosis, cardiac mapping, or the like.

Irrigated electrophysiology catheters are also known. An irrigated electrophysiology catheter is an electrophysiology catheter that is equipped to deliver an irrigation fluid, such as saline, to a location proximate the electrodes. The irrigation fluid serves, for example, to cool the electrodes or to disperse body fluids therefrom, to cool or bathe surrounding tissue, and/or to couple the electrodes to the tissue surface in the case of relatively highly conductive fluid(s).

In many irrigated electrophysiology catheters, a peristaltic pump is used to deliver the irrigation fluid. Typical peristaltic pumps operate by rotating a number of rollers mounted on a rotor to periodically compress an irrigation tube between the rollers and a pump housing or clamp, which forces the irrigation fluid through the irrigation tube.

It is known, however, that peristaltic pumps may generate electrical noise, which may undesirably couple to an electrogram signal measured by the electrodes on the irrigated electrophysiology catheter, to an electroanatomical visualization, localization and/or position system coupled to a subject, and/or to other diagnostic or therapeutic equipment. This noise is referred to herein as the “noise signal.”

### BRIEF SUMMARY OF THE INVENTION

It is therefore desirable to be able to provide a peristaltic pump that exhibits a reduced noise signal for use in connection with irrigated electrophysiology catheters.

The inventors have discovered that the peristaltic pump noise signal is synchronized with the peristaltic pump rollers lifting away from the irrigation tubing. That is, the noise signal “spikes” when the irrigation fluid moves out of the peristaltic pump and into the irrigated electrophysiology catheter. The inventors have surprisingly discovered that, by minimizing surface adhesion between the irrigation tubing and the pump clamp, this noise signal can be minimized.

Disclosed herein is an irrigation system for use with an irrigated electrophysiology catheter, including: a peristaltic pump including a pump clamp and a rotor, wherein the pump clamp and the rotor are spaced apart to provide a tubing channel therebetween; and an irrigation tube configured to be positioned between the pump clamp and the rotor within the tubing channel, wherein at least one of the pump clamp and the irrigation tube is treated to reduce surface adhesion therebetween. In some embodiments, an outer surface of the irrigation tube is distressed. In other embodiments, an outer wall of the tubing channel is distressed. In still other embodiments, a lubricant is interposed between the irrigation tube and the pump clamp. The lubricant may be applied to the irrigation tube and/or to the pump clamp.

Also disclosed herein is an irrigation system for use with an irrigated catheter, including: a peristaltic pump including a pump clamp and a rotor, wherein the pump clamp and the rotor define a tubing channel therebetween; and an irrigation tube configured to be positioned between the pump clamp and the rotor within the tubing channel, wherein at least one of the pump clamp and the irrigation tube is treated to minimize perturbations in pulsatile flow of an irrigation fluid through the irrigation tube. For example, at least one of the pump clamp and the irrigation tube can be treated to minimize surface adhesion between the pump clamp and the irrigation tube. In some embodiments, at least one of an outer surface of the irrigation tube and an outer wall of the tubing channel is distressed. In other embodiments, at least one of an outer surface of the irrigation tube and an outer wall of the tubing channel is lubricated.

In another aspect, the present disclosure relates to a method of reducing noise in an irrigated electrophysiology catheter system. The irrigated electrophysiology catheter system includes a peristaltic pump including a pump clamp, a rotor, and an irrigation tube positioned between the pump clamp and the rotor. The method includes treating at least one of the pump clamp and the irrigation tube to reduce surface adhesion therebetween.

In certain aspects of the disclosure, the step of treating involves distressing at least one of an outer surface of the irrigation tube and an inner surface of the pump clamp. For example, the inner surface of the pump clamp can be bead blasted and/or the outer surface of the irrigation tube can be sanded or etched. Alternatively, or additionally, at least one of an outer surface of the irrigation tube and an inner surface of the pump clamp can be lubricated with a lubricant (e.g., a liquid lubricant or a powder lubricant). The lubricant can also be impregnated into the irrigation tube and/or the pump clamp.

Advantageously, the present disclosure provides a peristaltic pump for use in connection with irrigated electrophysiology catheters that exhibits a reduced noise signal.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 schematically depicts a peristaltic pump.

FIG. 2 is a close-up schematic illustration of the interface between the rotor, the irrigation tube, and the pump clamp of the peristaltic pump shown in FIG. 1.

FIGS. 3A and 3B illustrate the noise signal of a conventional peristaltic pump.

FIGS. 4A and 4B illustrate the noise signal of a peristaltic pump according to the present disclosure.

### DETAILED DESCRIPTION OF THE INVENTION

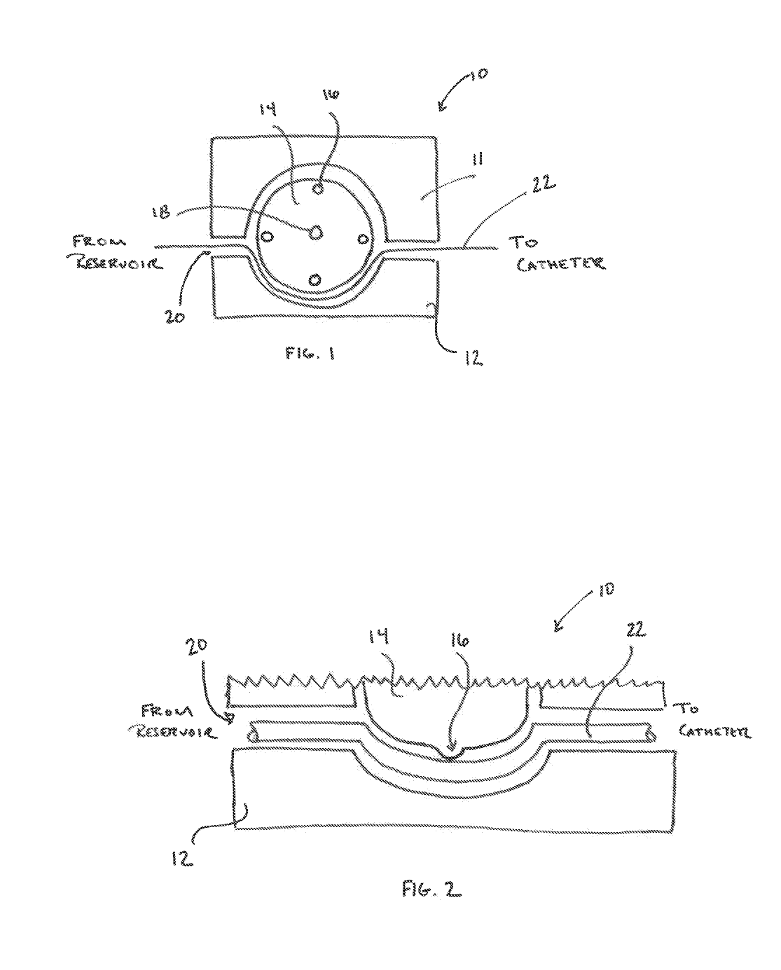

Referring now to the figures, and in particular to FIGS. 1 and 2, a peristaltic pump 10 is shown in schematic illustration. The configuration and operation of peristaltic pump 10 will be familiar to those of skill in the art, such that a detailed explanation thereof is not necessary herein. Instead, only those features of peristaltic pump 10 pertinent to understanding the present disclosure will be described below.

Peristaltic pump 10 generally includes a housing 11, a pump clamp 12, and a rotor 14. Rotor 14 includes a plurality of rollers 16 spaced about the circumference of rotor 14 and is mounted to rotate about an axle 18. Typically, although not necessarily, rollers 16 are evenly spaced about the circumference of rotor 14.

A tubing channel 20 is defined between pump clamp 12 and rotor 14. Tubing channel 20 accommodates an irrigation tube 22. One end of irrigation tube 22 can be coupled to a suitable reservoir of irrigation fluid (not shown), while the opposite end of irrigation tube 22 can be coupled to an irrigated electrophysiology catheter (not shown). Thus, when in operation, peristaltic pump 10 moves irrigation fluid from the reservoir into the electrophysiology catheter, where it moves through one or more irrigation lumens and exits via one or more irrigation ports.

As best shown in FIG. 2, a portion of irrigation tube 22 is positioned between pump clamp 12 and rotor 14. One of ordinary skill in the art will appreciate that, as rotor 14 turns, rollers 16 will periodically (if rollers 16 are evenly spaced around the circumference of rotor 14) impinge upon irrigation tube 22, pushing irrigation tube 22 against pump clamp 12 and forcing fluid through irrigation tube 22 to provide a pulsatile flow of irrigation fluid to the electrophysiology catheter.

When rollers 16 move off of irrigation tube 22, irrigation tube 22 springs back from pump clamp 12. As described above, the inventors have discovered that the peristaltic pump noise signal peaks when this occurs. The inventors have further discovered that, the more abruptly irrigation tube 22 springs back from pump clamp 12, the higher the amplitude of the noise signal peak will be.

It is believed that the pulsatile flow through irrigation tube 22 perturbs the electrical charge layer (or “double layer”) that exists at the interface between the irrigation fluid and the irrigated electrode on the electrophysiology catheter (the “electrode-electrolyte interface”). The double layer can be viewed as an electrical boundary layer, and gives rise to a differential capacitance at the electrode-electrolyte interface. The differential capacitance, C, is defined by the following formula:

$$C = \frac{d σ}{d Ψ} ,$$

where σ is surface charge and Ψ is surface potential.

The inventors hypothesize and recognize that the pulsatile flow of the irrigation fluid perturbs the double layer in a manner akin to surface renewal in interfacial fluid flow and mass transport phenomenon. Thus, the flow variations inherent in the pulsatile flow of a peristaltic pump dynamically affect the differential capacitance at the electrode-electrolyte interface, which then distorts the electrical signal that is being measured by the electrode.

Accordingly, it is desirable to minimize the abruptness with which irrigation tube 22 springs back from pump clamp 12, thereby minimizing perturbations in the flow of irrigation fluid and the associated disruptions in the double layer at the electrode-electrolyte interface, which in turn minimizes the amplitude of the peristaltic pump noise signal. According to the present disclosure, this is accomplished by treating at least one of irrigation tube 22 and pump clamp 12 to reduce surface adhesion between irrigation tube 22 and pump clamp 12.

Thus, according to a first aspect of the disclosure, pump clamp 12 is treated to reduce surface adhesion with irrigation tube 22. For example, the surface of pump clamp 12 that contacts irrigation tube 22 (which can interchangeably be referred to as the “inner surface of the pump clamp” or the “outer wall of the tubing channel”) can be distressed (e.g., roughened). One suitable method of distressing pump clamp 12 is by bead blasting, such as by using an aluminum oxide bead, having a grit size of about 60, with the air pressure of the bead blaster set to about 60 psi.

In another aspect of the disclosure, irrigation tube 22 is treated to reduce surface adhesion with pump clamp 12. For example, the outer surface of irrigation tube 22 can be distressed (e.g., roughened), such as by sanding or etching (e.g., chemical etching).

Of course, it is also contemplated that both pump clamp 12 and irrigation tube 22 can be distressed.

In additional aspects of the disclosure, irrigation tube 22 and/or pump clamp 12 are lubricated in order to reduce surface adhesion therebetween. For example, a liquid or powdered lubricant (e.g., baby powder, corn starch or the like) can be interposed between pump clamp 12 and irrigation tube 22 within tubing channel 20. Alternatively, the lubricant can be applied directly to irrigation tube 22 and/or pump clamp 12, for example by impregnating irrigation tube 22 and/or pump clamp 12 with a suitable lubricant.

The treatments disclosed herein reduce the contact surface area between pump clamp 12 and irrigation tube 22, and thus the surface adhesion between pump clamp 12 and irrigation tube 22. Advantageously, therefore, the treatments disclosed herein allow irrigation tube 22 to spring back from pump clamp 12 less abruptly, which results in less perturbation of the flow of irrigation fluid through an irrigated electrophysiology catheter. This, in turn, desirably reduces the amplitude of the peristaltic pump noise signal.

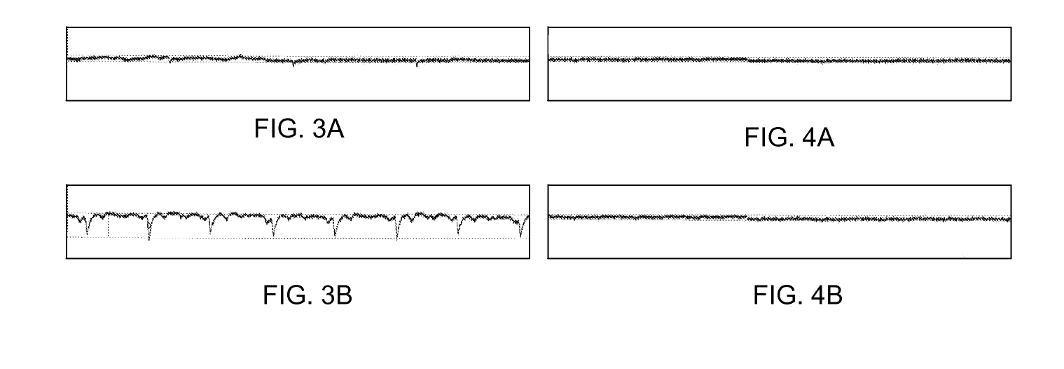

FIGS. 3A and 3B illustrate the noise signal of a conventional peristaltic pump at two different flow rates: 10 ml/min and 20 ml/min. The advantages of this disclosure are illustrated in FIGS. 4A and 4B. These figures correspond to FIGS. 3A and 3B, except FIGS. 4A and 4B illustrate the noise signal at two different flow rates through a peristaltic pump according to the present disclosure. The reduction in the noise signal resulting from applying the teachings herein is apparent.

Although several embodiments of this disclosure have been described above with a certain degree of particularity, those skilled in the art could make numerous alterations to the disclosed embodiments without departing from the spirit or scope of this disclosure. For example, the term “treating” is intended to be interpreted broadly to encompass any modification to the pump clamp and/or the irrigation tube that beneficially reduces the surface adhesion therebetween. Likewise, the term “distressing” is intended to be interpreted broadly to encompass any mechanical or chemical modification to the pump clamp and/or the irrigation tube that beneficially reduces the surface adhesion therebetween.

All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and can include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure can be made without departing from the spirit of the disclosure as defined in the appended claims.

1. An irrigation system for use with an irrigated electrophysiology catheter, comprising:
a peristaltic pump including a pump clamp and a rotor, wherein the pump clamp and the rotor are spaced apart to provide a tubing channel therebetween; and
an irrigation tube configured to be positioned between the pump clamp and the rotor within the tubing channel,
wherein at least one of the pump clamp and the irrigation tube is treated to reduce surface adhesion therebetween.

2. The irrigation system according to claim 1, wherein an outer surface of the irrigation tube is distressed.

3. The irrigation system according to claim 1, wherein an outer wall of the tubing channel is distressed.

4. The irrigation system according to claim 1, further comprising a lubricant interposed between the irrigation tube and the pump clamp.

5. The irrigation system according to claim 4, wherein the lubricant is applied to the irrigation tube.

6. The irrigation system according to claim 4, wherein the lubricant is applied to the pump clamp.

7. An irrigation system for use with an irrigated catheter, comprising:
a peristaltic pump including a pump clamp and a rotor, wherein the pump clamp and the rotor define a tubing channel therebetween; and
an irrigation tube configured to be positioned between the pump clamp and the rotor within the tubing channel,
wherein at least one of the pump clamp and the irrigation tube is treated to minimize perturbations in pulsatile flow of an irrigation fluid through the irrigation tube.

8. The irrigation system according to claim 7, wherein at least one of the pump clamp and the irrigation tube is treated to minimize surface adhesion between the pump clamp and the irrigation tube.

9. The irrigation system according to claim 7, wherein at least one of an outer surface of the irrigation tube and an outer wall of the tubing channel is distressed.

10. The irrigation system according to claim 7, wherein at least one of an outer surface of the irrigation tube and an outer wall of the tubing channel is lubricated.

11. A method of reducing noise in an irrigated electrophysiology catheter system, comprising conditioning a peristaltic pump including a pump clamp, a rotor, and an irrigation tube positioned between the pump clamp and the rotor by treating at least one of the pump clamp and the irrigation tube to reduce surface adhesion therebetween.

12. The method according to claim 11, wherein treating comprises distressing at least one of an outer surface of the irrigation tube and an inner surface of the pump clamp.

13. The method according to claim 12, wherein distressing comprises bead blasting the inner surface of the pump clamp.

14. The method according to claim 12, wherein distressing step comprises sanding the outer surface of the irrigation tube.

15. The method according to claim 12, wherein distressing step comprises etching the outer surface of the irrigation tube.

16. The method according to claim 11, wherein treating step comprises lubricating at least one of an outer surface of the irrigation tube and an inner surface of the pump clamp with a lubricant.

17. The method according to claim 16, wherein the lubricant comprises a liquid lubricant.

18. The method according to claim 16, wherein the lubricant comprises a powder lubricant.

19. The method according to claim 16, wherein the irrigation tube is impregnated with the lubricant.

20. The method according to claim 16, wherein the pump clamp is impregnated with the lubricant.
